(19) United States
(12) Patent Application Publication
(10) Pub. No.: US 2005/0249762 A1
(43) Pub. Date: Nov. 10, 2005

(54) Zinc calcium aluminum borosilicate

(76) Inventors: Dennis Dean Loyd, West Chester, OH (US); Larry Lee Lough, Springdale, OH (US)

Correspondence Address: Schwegman, Lundberg, Woessner & Kluth, P.A., P.O. Box 2938, Minneapolis, MN, 55402, US

(21) Appl. No.: 11/100,357

(22) Filed: Apr. 6, 2005

Related U.S. Application Data
(63) Continuation-in-part of application No. 10/701,875, filed on Nov. 5, 2003.
(63) Continuation-in-part of application No. 10/973,819, filed on Oct. 26, 2004.

(51) Int. Cl. A61K 7/42

(52) U.S. Cl. 424/401

(57) ABSTRACT

Cosmetic mixtures made with zinc calcium aluminum borosilicate are provided. The zinc calcium aluminum borosilicate is made as glass and crushed into particles having small diameter sizes. The particles make up a powder that is then mixed with cosmetic ingredients to produce modified cosmetic products.

### RELATED APPLICATIONS

The present application is a Continuation-In-Part of U.S. Ser. No. 10/701,875, entitled: “Ultraviolet Absorption and Radiation Shielding for Raw Materials and Products,” filed on Nov. 5, 2003; the disclosure of which is incorporated by reference herein. Moreover, the present application is a Continuation-In-Part of U.S. Ser. No. 10/973,819, entitled “Ultraviolet Absorption,” filed on Oct. 26, 2004; the disclosure of which is also incorporated by reference herein.

### FIELD OF THE INVENTION

The present invention is related to zinc calcium aluminum borosilicate mixtures, and more particularly to cosmetics modified with zinc calcium aluminum borosilicate mixtures.

### BACKGROUND OF THE INVENTION

Cosmetic products include a variety of modifiers for a variety of purposes. For example, ultraviolet (UV) modifiers may supply some degree of UV protection to cosmetic products. Vitamin modifiers offer purported benefits to the skin or hair to which the cosmetic product is applied. Other modifiers provide texture, flow, and color to the cosmetic products for purposes of making the cosmetic products feel and appear agreeable to a consumer.

UV protection is of particular importance to cosmetic manufacturers and consumers in view of recent studies related to UV exposure and unusual incidences of skin cancer. However, some UV absorbers provide only marginal UV protection while the expense associated with adding the UV absorbers is fairly significant for the manufacturer.

Moreover, most sunscreens protect against primarily UVB radiation but not UVA radiation. UVB is radiation from the sun that is most noticeable to the consumer because excessive exposure results in the burning and tanning of the skin. However, recent studies have indicated that UVA radiation is associated with long term skin damage even though its effects are not immediately noticeable by individuals. In general, most sunscreens and other cosmetic additives provide very little UVA protection and tend to focus exclusively on UVB protection, since this is the type of protection most readily apparent to a consumer.

Therefore, improved UVA and UVB protection for cosmetics is desirable, such that the resulting cosmetics are not excessively expensive to manufacture and provide increased UV protection for cosmetic products.

### SUMMARY OF THE INVENTION

Briefly and in general terms, a cosmetic mixture is provided. The cosmetic mixture includes approximately 30-60 percent of silicon oxide, approximately 10-40 percent of zinc oxide, approximately 2-20 percent of aluminum oxide, approximately 5-20 percent of calcium oxide, and approximately 2-10 percent of boric oxide.

### BRIEF DESCRIPTION OF THE DRAWINGS

FIG. 1 is a diagram of cosmetic mixture, according to an example embodiment.

FIG. 2 is a diagram of another cosmetic mixture, according to an example embodiment.

FIG. 3 is a diagram of a method for creating a zinc calcium aluminum borosilicate modified cosmetic product, according to an example embodiment.

### DETAILED DESCRIPTION OF THE INVENTION

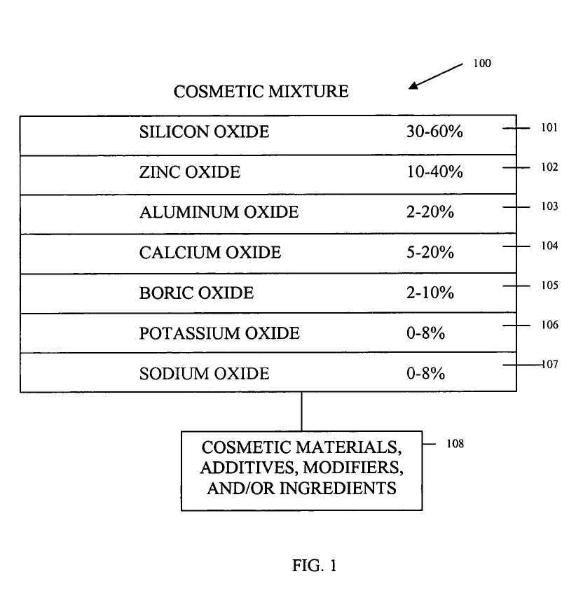

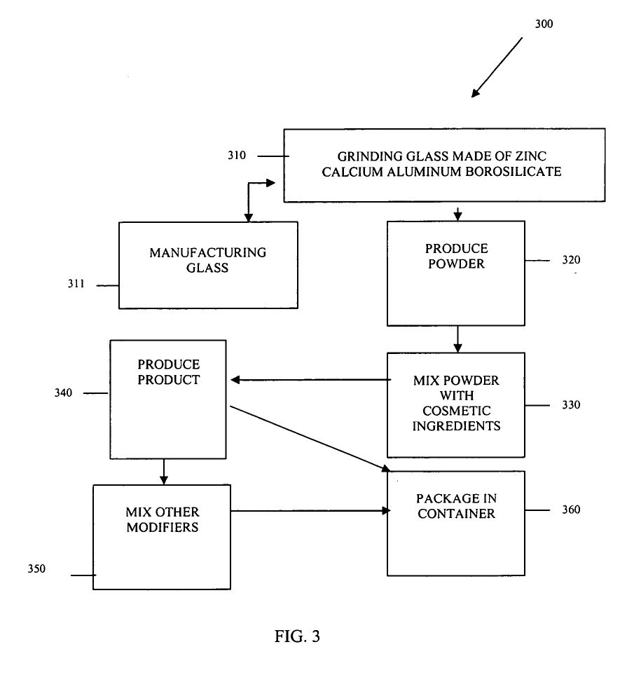

FIG. 1 illustrates a diagram for a cosmetic mixture 100, according an example embodiment. The ingredients of the cosmetic mixture 100 are derived from a novel blend of glass. In an embodiment, the glass is specifically manufactured to acquire the unique composition and mixture presented in FIG. 1. The glass is then milled or ground to particular sizes that are substantially irregular in shape and have diameter sizes of less than or equal to approximately 15 microns. At these small sizes, the glass is safe for inclusion in cosmetic materials and for human exposure and consumption. Specific methods or techniques for manufacturing the glass and integrating the glass into cosmetics are discussed in greater detail below with the discussion of FIG. 3.

The cosmetic mixture 100 includes approximately 30-60 percent of silicon oxide 101, approximately 10-40 percent of zinc oxide 102, approximately 2-20 percent of aluminum oxide 103, approximately 5-20 percent of calcium oxide 104, and approximately 2-10 percent of boric oxide 105. In an embodiment, the cosmetic mixture 100 may also include approximately 0-8 percent potassium oxide 106, and/or approximately 0-8 percent sodium oxide 107. The cosmetic mixture 100 may be referred to as a novel composition referred to as zinc calcium aluminum borosilicate.

In an embodiment, the cosmetic mixture 100 also include a variety of other cosmetic materials, additives, modifiers, and/or ingredients 108. For example, ingredients such as oils, esters, cosmetic carriers, vitamins, enzymes, softening agents, moisturizing agents, surfactants, perfumes, alcohols, gelling agents, waxes, stabilizers, emulsifying agents, pigments, preservatives, anti-inflammatory substances, antioxidants, bulking agents, colorants, and other ingredients typically used in cosmetic formulations as is known to one of ordinary skill in the cosmetic arts. Thus, the cosmetic mixture 100 may be mixed with raw materials associated with cosmetics or additives associated with cosmetic products.

Some example mixtures between the cosmetic mixture 100 and cosmetic ingredients are now provided for purposes of illustration. These example composite mixtures are not intended to limit applications of the novel cosmetic mixture 100; rather, the composite mixtures are presented for purposes of understanding different applications and composite cosmetic materials that can be created with the teachings presented herein. Again, methods of manufacturing the composite materials are supplied herein and below with the discussion of FIG. 3.

An example makeup foundation may include the following ingredients presented in Table 1:

Zinc Calcium Aluminum Borosilicate - cosmetic mixture 100
Water
Squalane
Color pigments
Steareth-21
Steareth-2
C12-15 alkyl benzoate
Stearyl alcohol
PEG-8
Silicone-cyclomethicone
Preservative
Perfume Oil

Another example makeup foundation may be as depicted in Table 2:

Zinc Calcium Aluminum Borosilicate - cosmetic mixture 100
Water
Colorants
Steareth-21
Steareth-2
C12-15 alkyl benzoate
Stearyl alcohol
PEG-8
Silicone-cyclomethicone
Honey Extract

An example peeling gel may appear as depicted in Table 3:

Zinc Calcium Aluminum Borosilicate - cosmetic mixture 100
Water
Carbomer
Glycerine
Propylene glycol
Triethanolamine
Silicone
Preservative
Perfume oil

Another example body gel may appear as depicted in Table 4:

Zinc Calcium Aluminum Borosilicate - cosmetic mixture 100
Water
Glycerine
Crosspolymer
Octyl salicylate
Benzophenone
Dimethicone
Jojoba oil
Triethanolamine
Preservative
Perfume oil

Still another body or skin gel may appear as depicted in Table 5:

Zinc Calcium Aluminum Borosilicate - cosmetic mixture 100
Water
Carbomer
Glycerine
Triethanolaniine
Preservative
Perfume oil

An example facial mask may appear as depicted in Table 6:

Zinc Calcium Aluminum Borosilicate - cosmetic mixture 100
Water
Carbomer
Glycerine
Propylene glycol
Kaolin
Glyceryl stearate
Stearic acid
Jojoba oil
Cyclomethicone
Triethanolamine
Silicone-cyclomethicone
Preservative
Perfume oil

Still another example cosmetic as a skin emulsion may appear as depicted in Table 7:

Zinc Calcium Aluminum Borosilicate - cosmetic mixture 100
Water
Cetearyl alcohol
Isopropyl myristate
Dimethicone
PEG 40 castor oil
Preservative
Perfume oil

The example cosmetics may also include sunscreens that are mixed with the cosmetic mixture 100. A variety of sunscreens may be used; some example sunscreens are included in U.S. Pat. Nos. 5,087,445 and 5,073,372; the disclosures of which are incorporated by reference herein.

For example, some sunscreens that may be formulated with the cosmetic mixture 100 may be selected from aminobenzoic acid, amyldimethyl PABA, cincoxate, diethanolamine p-methoxycinnamate, digalloyl trioleate, dioxybenzone, 2-ethoxyethyl p-methoxycinnamate, ethyl 4-bis(hydroxypropyl) amino benzoate, 2-ethylhexyl-2-cyano-3,3-diphenylacrylate, ethylhexyl p-methoxycinnamate, 2-ethylhexyl salicylate, Glyceryl amino benzoate, homomenthyl salicylate, homosalate, 3-imidazol-4-ylacrylic acid and ethyl ester, methyl anthranilate, octyldimenthyl PABA, 2-phenylbenzimidazole-5-sulfonic acid and salts, red petrolatum, sulisobenzone, titanium dioxide, Triethanolamine salicylate, N,N, N-trimethyl-4-(2-oxoborn-3-ylidene methyl) anillinium methyl sulfate, and various mixtures thereof.

Additionally sunscreens may include a UV-photo protecting agent, such as dibenzoylmethane sunscreen avobenzone or 4-(tert-butyl)-4′-methooxyibenzoylmethane, which is well known to those of ordinary skill in the cosmetic arts. Furthermore, the sunscreens may be physical blockers that reflect or scatter UV radiation or chemical absorbers that absorb harmful radiation. The chemical absorbers may protect against UVA or UVB radiation.

According to an embodiment, the cosmetic mixture 100 is a homogeneous mixture; meaning that the ingredients and the components of the cosmetic mixture 100 are uniformly dispersed throughout the cosmetic mixture 100.

The phasing of mixing the cosmetic mixture 108 with cosmetic materials, additives, modifiers, and/or ingredients 108 may be any used and recognized by those of ordinary skill in the cosmetic arts.

In fact, it is noted and appreciated that any existing cosmetic formulation may be modified and mixed with the cosmetic mixture 100 for purposes of producing an improved UV cosmetic product. The cosmetic mixture 100 is inexpensive to produce and to integrate with existing fabrication processes associated with cosmetics. A cosmetic as any aerosol, gel, paste, powder, liquid, etc. that is applied to a consumer's body and/or hair.

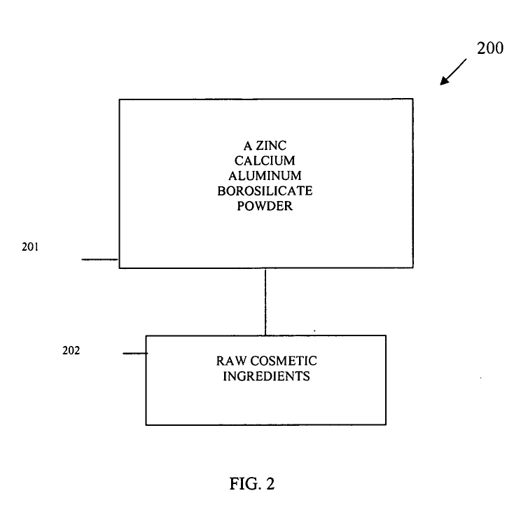

FIG. 2 is a diagram of another cosmetic mixture 200, according to an example embodiment. In an embodiment, at least a portion of the cosmetic mixture 200 is the cosmetic mixture 100 depicted and described above with respect to FIG. 1.

The cosmetic mixture 200 includes a zinc calcium aluminum borosilicate powder 201 and raw cosmetic ingredients 202. The zinc calcium aluminum borosilicate powder 201 is mixed and integrated with the raw cosmetic ingredients 202. According to an embodiment, the composition and percentage weight of ingredients within the zinc calcium aluminum borosilicate powder 201 is the cosmetic mixture 100 presented above with respect to FIG. 1.

Furthermore, and in an embodiment, the zinc calcium aluminum borosilicate powder 201 is derived from virgin glass manufactured for purposes of being a cosmetic filler material. The virgin glass is then ground into substantially acicular irregular shaped particles having individual diameter sizes of approximately less than or equal to 15 microns. Techniques for producing the zinc calcium aluminum borosilicate powder 201 are presented below with respect to the discussion associated with FIG. 3.

The raw cosmetic ingredients 202 can include any type of cosmetic product and its ingredients, modifiers, additives, and/or other fillers. Some example cosmetic formulations and example cosmetic products were presented above with respect to FIG. 1. The form of a cosmetic may be a gel, a powder, a paste, an aerosol, a liquid, a wax, etc. It is to be understood that any readily known cosmetic (known by one of ordinary skill in the cosmetic arts) formulation and manufacturing process may be augmented with the zinc calcium aluminum borosilicate powder 201, and any such combination is included to fall within the scope of the embodiments for this invention.

FIG. 3 is a diagram of a method 300 for creating a zinc calcium aluminum borosilicate modified cosmetic product, according to an example embodiment. The method 300 may be implemented using devices and/or equipment available and known to one of ordinary skill in the glass and cosmetic arts.

At 310, glass made of zinc calcium aluminum borosilicate is ground or milled to small diameter sizes or into a powder which is suitable for integration with traditional cosmetic ingredients for traditional cosmetic products. In an embodiment, the zinc calcium aluminum borosilicate includes the ingredients and percentage weights of the ingredients which are depicted above with respect to the cosmetic mixture 100 of FIG. 1.

In an embodiment, at 311, the zinc calcium aluminum borosilicate glass may be manufactured in the following manner before it is ground into a powder at 310. All the materials such as silicon oxide, zinc oxide, aluminum oxide, calcium oxide, boric oxide, and optionally potassium oxide and/or sodium oxide are introduced into a ball mill and ground to 100 mesh particle sizes to provide an optimal mixture and reaction environment. Next, the materials are loaded into an oven and oxygen is purged with a gas mixture of approximately 90% nitrogen and 10% argon. It should be noted that the glass may be formed in various other reaction atmospheres known to one of ordinary skill in the art. The gas mixture is kept continuous as the heat up cycle begins. The melting is done by reducing the gas conditions. The oven is heated to approximately 800-1500 degrees Celsius until a molten state is achieved. At this point conditions are held for approximately 8 hours and checks are made for clarity of the glass mixture and full solution and percentage weights of the oxides. Once a melt has been completed, the glass mixture is quenched in a water bath or through chilled rollers. The glass mixture is then dried through a belt drier and fed into a continuous ceramic ball mill where it is ground into a powder and screen sifted through a 400 mesh SWECO vibrating classifier. At this point a novel zinc calcium aluminum borosilicate powder is available for use to be integrated with cosmetic ingredients.

At 320, a powder of the zinc calcium aluminum borosilicate is produced from the grinding process. The zinc calcium aluminum borosilicate powder may then be packaged and shipped to cosmetic manufacturers for integration into their cosmetic formulation processes.

Alternatively, at 330, the zinc calcium aluminum borosilicate powder may be directly fabricated into a process phasing of a cosmetic product, such that the zinc calcium aluminum borosilicate powder is mixed with raw cosmetic ingredients. This results, at 340, in a novel cosmetic product being produced having the zinc calcium aluminum borosilicate powder intimately integrated therein with the original cosmetic ingredients. Thus, a novel modified cosmetic product is produced, at 340.

In an embodiment, at 350, the modified cosmetic product may be further modified or altered with a variety of additional modifiers, additives, etc. Some example modifiers and additives were presented above with respect to FIG. 1.

At 370, the modified cosmetic product may be packaged in a product container and shipped to distributors, retail stores, or directly to consumers. These product containers may advertise that the cosmetic product includes a novel modified cosmetic product having unique UVA and/or UVB protection. Moreover, the method 300 is easily integrated with existing cosmetic fabrication and phasing processes in a more cost effective manner than what has been achievable in the past, since the zinc calcium aluminum borosilicate powder is produced and manufactured in a manner where acicular irregular shaped particles are produced.

It is now apparent to one of ordinary skill in the cosmetic arts how improved cosmetic products may be cost-effectively manufactured with zinc calcium aluminum borosilicate powder or mixtures. Furthermore, the resulting novel cosmetic products exhibit improved UVA and UVB protection.

Although specific embodiments have been illustrated and described herein, those of ordinary skill in the art will appreciate that any arrangement calculated to achieve the same purpose can be substituted for the specific embodiments shown. This disclosure is intended to cover any and all adaptations or variations of various embodiments of the invention. It is to be understood that the above description has been made in an illustrative fashion, and not a restrictive one. Combinations of the above embodiments, and other embodiments not specifically described herein will be apparent to one of ordinary skill in the art upon reviewing the above description. The scope of various embodiments of the invention includes any other applications in which the above structures and methods are used. Therefore, the scope of various embodiments of the invention should be determined with reference to the appended claims, along with the full range of equivalents to which such claims are entitled.

It is emphasized that the Abstract is provided to comply with 37 C.F.R. § 1.72(b) requiring an Abstract that will allow the reader to quickly ascertain the nature and gist of the technical disclosure. It is submitted with the understanding that it will not be used to interpret or limit the scope or meaning of the claims.

In the foregoing Detailed Description, various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate preferred embodiment.

1. A cosmetic mixture, including:
silicon oxide comprising approximately between 30-60 percent of the mixture;
zinc oxide comprising approximately between 1040 percent of the mixture;
aluminum oxide comprising approximately between 2-20 percent of the mixture;
calcium oxide comprising approximately between 5-20 percent of the mixture; and
boric oxide comprising approximately between 2-10 percent of the mixture.

2. The cosmetic mixture of claim 1 further including at least one of:
potassium oxide comprising approximately between 0-8 percent of the mixture; and
sodium oxide comprising approximately between 0-8 percent of the mixture.

3. The cosmetic mixture of claim 1, wherein the mixture is a powder having substantially acicular irregularly shaped particles.

4. The cosmetic mixture of claim 1 further including at least one:
oils;
esters;
cosmetic carriers;
vitamins;
enzymes;
softening agents;
moisturizing agents;
surfactants;
perfumes;
alcohols;
gelling agents;
waxes;
stabilizers;
emulsifying agents;
pigments;
preservatives;
anti-inflammatory substances;
antioxidants;
bulking agents; and
colorants.

5. The cosmetic mixture of claim 1, wherein the mixture is a homogeneous mixture.

6. The cosmetic mixture of claim 1 further including raw materials for a cosmetic product mixed with the mixture.

7. The cosmetic mixture of claim 1, wherein individual particles of the mixture have diameter sizes of less than approximately 15 microns within the mixture.

8. A cosmetic mixture, including:
a zinc calcium aluminum borosilicate powder; and
raw cosmetic ingredients mixed with the powder.

9. The cosmetic mixture of claim 8, wherein the powder is derived from virgin glass, and wherein each particle of the virgin glass has a diameter size of less than approximately 15 microns.

10. The cosmetic mixture of claim 8, wherein the particles are substantially irregular in shape.

11. The cosmetic mixture of claim 8, wherein the cosmetic mixture is at least one of a gel, a powder, a paste, an aerosol, a liquid, and a wax.

12. The cosmetic mixture of claim 8, wherein the zinc calcium aluminum borosilicate powder includes approximately between 10-40 percent zinc oxide.

13. The cosmetic mixture of claim 12, wherein the zinc calcium aluminum borosilicate powder includes approximately between 30-60 percent silicon oxide.

14. The cosmetic mixture of claim 13, wherein the zinc calcium aluminum borosilicate powder includes approximately 5-20 percent calcium oxide.

15. The cosmetic mixture of claim 14, wherein zinc calcium aluminum borosilicate powder includes approximately 2-20 percent aluminum oxide.

16. The cosmetic mixture of claim 15, wherein the zinc calcium aluminum borosilicate powder includes approximately 2-10 percent boric oxide.

17. A method, comprising:
grinding glass made of zinc calcium aluminum borosilicate into particle sizes of less than approximately 15 microns in diameter to produce a powder; and
mixing the powder with cosmetic ingredients to produce a modified cosmetic product.

18. The method of claim 17 further comprising, packaging the cosmetic product in a product container for transport.

19. The method of claim 17 further comprising, manufacturing the glass before grinding the glass.

20. The method of claim 17 further comprising, mixing other modifiers with the modified cosmetic product.
